(19) United States
(12) Patent Application Publication
(10) Pub. No.: US 2010/0151473 A1
(43) Pub. Date: Jun. 17, 2010

(54) METHODS AND COMPOSITIONS FOR HYBRIDIZING NUCLEIC ACIDS

(76) Inventors: Joanne M. Yeakley, Encinitas, CA (US); Ross Edward Lenta, San Diego, CA (US); Min-Jui Richard Shen, Poway, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/634,531

(22) Filed: Dec. 9, 2009

Related U.S. Application Data
(60) Provisional application No. 61/121,501, filed on Dec. 10, 2008.

(51) Int. Cl. C12Q 1/68; C12P 19/34

(52) U.S. Cl. 435/6; 435/91.1

(57) ABSTRACT

Methods and compositions for hybridizing nucleic acids are disclosed herein. Also disclosed herein are methods and compositions to detect a nucleic acid provided to an array.

### REFERENCE TO RELATED APPLICATIONS

This is a non-provisional application that claims priority to U.S. Provisional Application No. 61/121,501, filed Dec. 10, 2008, which is incorporated herein by reference in its entirety.

### FIELD OF THE INVENTION

The present invention relates to the fields of biochemistry and molecular biology. More specifically, the present invention relates to methods and compositions for hybridizing nucleic acids.

### BACKGROUND

Nucleic acid hybridization occurs when double-stranded nucleic acids are formed from single-stranded nucleic acids by interaction of complementary base pairs on the respective nucleic acid strands. Nucleic acids are molecules that can be made up of the nucleosides adenosine (A), guanosine (G), cytidine (C), thymidine (T), uridine (U), as well as a variety of other modified and non-naturally-occurring nucleosides. In nature, nucleosides are covalently linked by phosphate ester linkages between the 3′-hydroxyl group on the sugar residue of one nucleoside and the phosphate linked to the 5′ position of the adjacent nucleoside. In DNA, the sugar residue is deoxyribose, while in RNA, the sugar residue is ribose. There is chemical affinity between respective pairs of purine and pyrimidine bases comprising part of each nucleotide unit, for example, T-A, C-G, and A-U. Hybridization is facilitated by the presence of complementary sequences of bases in nucleic acids, and can be an extremely specific and sensitive reaction under certain conditions. Moreover, nucleic acid hybridization is often a methodology employed in the use of nucleic acid-based microarrays.

Microarrays have become an increasingly important tool in medicine, biotechnology and related fields. A microarray typically includes a support that contains numerous capture probes. These capture probes are usually selected for their binding affinity towards their target in a sample presented to the array. After applying the sample to the array the interaction between probes on the support and its corresponding target can be observed through various labeling and detection techniques, thereby providing qualitative and quantitative data about the target in the tested sample. Microarray technology has been applied to many types of molecules, including nucleic acids, proteins, and other chemical compounds. Nucleic acid microarrays can provide, for example, a means to analyze the expression of many different genes in a sample simultaneously. While microarrays are emerging as a mature tool, challenges to improve microarray technology remain. Accordingly, there is a continued interest in developing systems and methods to provide more efficient and less expensive microarray tools.

### BRIEF DESCRIPTION OF THE DRAWING

FIG. 1 shows the results of hybridizing nucleic acids in the presence of various divalent cations.

### SUMMARY OF THE INVENTION

Some embodiments of the present invention relate to methods for hybridizing nucleic acids. Such methods can include obtaining a buffered solution containing a double-stranded nucleic acid; converting the double-stranded nucleic acid to a single-stranded nucleic acid in the buffered solution; and hybridizing the single-stranded nucleic acid to a capture probe in the buffered solution. In some embodiments, the double-stranded nucleic acid can include DNA.

Some of the hybridization methods described herein utilize one or more nucleases to convert the double-stranded nucleic acid to a single-stranded nucleic acid prior to hybridization. Such methods include the step of digesting the double-stranded nucleic acid with the one or more nucleases. In some embodiments, the one or more nuclease can include, but are not limited to, exonuclease III, T7 exonuclease, lambda exonuclease and combinations of such nucleases. In preferred embodiments, the one or more nucleases comprise lambda exonuclease.

Other hybridization methods described herein relate to converting the double-stranded nucleic acid to a single-strand, and then hybridizing the single strand to a capture probe on an array in a single, or the same, buffered solution. In some such methods, the buffered solution can have a pH of at least 7.5. In certain methods, the buffered solution comprises Tris buffer. In preferred methods, the volume of the buffered solution is not substantially or significantly changed between the conversion step and the hybridization step. In still more preferred methods, neither the volume of the buffered solution nor the concentration of ions or other charged molecules in the buffered solution is substantially or significantly changed between the conversion and hybridization steps. In some methods, the buffered solution lacks a concentration of monovalent cations sufficient to substantially inhibit the activity of the one or more nucleases used to convert the double-stranded nucleic acid to a single-stranded nucleic acid. In other embodiments of the disclosed methods, the buffered solution essentially lacks monovalent cations. In still other methods, the buffered solution lacks a concentration of phosphate ions sufficient to substantially inhibit the activity of said nuclease. In preferred embodiments, the buffered solution essentially lacks phosphate ions.

In some preferred methods for hybridizing nucleic acids, the buffered solution comprises one or more divalent cations. In such embodiments, the one or more divalent cations can be present in the buffered solution at a concentration sufficient both to promote activity of the one or more exonucleases used in the conversion step and to permit hybridization of the single-stranded nucleic acid complementary to the capture probe. In some of these embodiments, the one or more divalent cations include, but are not limited to, Mg²⁺, Mn²⁺, Zn²⁺ or combinations thereof. In still other embodiments, the buffered solution can comprise one or more polyamines. In some embodiments, the one or more polyamines can be used in place of, or in addition to, the divalent cations.

Some methods for hybridizing nucleic acids include a step of hybridizing the single-stranded nucleic acid to a capture probe. In such methods, the capture probe can be associated with a solid support. Although the solid support can be essentially any size and/or shape, in preferred embodiments, the solid support is a planar surface. In other embodiments, the solid support can be a fiber optic bundle. In still other embodiments, the solid support comprises one or more microspheres. Microspheres can be of any shape, size or construction. For example, the microsphere can be subnanometer to millimeter size; isometrical to elongated; with or without surface features; or solid or porous. Microspheres may or may not be associated with another surface, such as a planar array, fiber optic substrate or microtiter plate.

In the methods for hybridizing nucleic acids described herein, the capture probe can be one of a plurality of different capture probes. In some of the hybridization methods, the capture probes are distributed on the surface of a substrate. For example, the capture probes can be orderly distributed or randomly distributed on the substrate. In other methods, the capture probes can be distributed randomly on the substrate. Some methods for hybridizing nucleic acids further include a step of extending the 3′ end of the capture probe by providing a polymerase enzyme. In some embodiments, the hybridization methods described herein are employed as one or the early steps in an array-based sequencing method.

In addition to the methods for hybridizing nucleic acids described herein, also provided are methods for detecting the presence of a nucleic acid complementary to a capture probe. Such methods can include obtaining a buffered solution comprising a double-stranded nucleic acid; converting the double-stranded nucleic acid to a single-stranded nucleic acid in the buffered solution; providing the single-stranded nucleic acid to a capture probe or plurality of different capture probes in the buffered solution; allowing the single-stranded nucleic acid to hybridize to a capture probe having sufficient complementary to permit hybridization under the conditions used for hybridization; and determining whether the single-stranded nucleic acid hybridizes to a capture probe. Hybridization of the single-stranded nucleic acid to the capture probe indicates the presence of a nucleic acid having complementary to the capture probe. In some embodiments, the double-stranded nucleic acid used in the above process is DNA such as, genomic DNA or a fragment of genomic DNA.

Some of the methods described herein for detecting the presence of a nucleic acid complementary to a capture probe include converting the double-stranded nucleic acid to a single-stranded nucleic by using one or more nucleases. The one or more nucleases can include, but are not limited to, exonuclease III, T7 exonuclease, lambda exonuclease or combinations of such nucleases. In preferred embodiments, the one or more nucleases comprise lambda exonuclease.

In particular methods for detecting the presence of a nucleic acid complementary to a capture probe, the buffered solution can have a pH of at least 7.5. In certain methods, the buffered solution comprises Tris buffer. As with the hybridization methods described herein, preferred embodiments of the detection methods relate to methods where the volume of the buffered solution in not substantially or significantly changed between the conversion step and the hybridization step. In still more preferred methods, neither the volume of the buffered solution nor the concentration of ions or other charged molecules in the buffered solution is substantially or significantly changed between the conversion and hybridization steps. In some methods, the buffered solution lacks a concentration of monovalent cations sufficient to substantially inhibit the activity of the one or more nucleases used to convert the double-stranded nucleic acid to a single-stranded nucleic acid. In more embodiments of the disclosed methods, the buffered solution essentially lacks monovalent cations. In still more methods, the buffered solution lacks a concentration of phosphate ions sufficient to substantially inhibit the activity of said nuclease. In preferred embodiments, the buffered solution essentially lacks phosphate ions.

In some preferred methods for detecting the presence of a nucleic acid complementary to a capture probe, the buffered solution comprises one or more divalent cations. In such embodiments, the one or more divalent cations can be present in the buffered solution at a concentration sufficient both to promote activity of the one or more exonucleases used in the conversion step and to permit hybridization of the single-stranded nucleic acid complementary to the capture probe. In some of these embodiments, the one or more divalent cations include, but are not limited to, Mg²⁺, Mn²⁺, Zn²⁺ or combinations thereof. In still other embodiments, the buffered solution can comprise one or more polyamines. In some embodiments, the one or more polyamines can be used in place of, or in addition to, the divalent cations.

In some methods for detecting the presence of a nucleic acid complementary to a capture probe, the capture probe is associated with a solid support. Although the solid support can be essentially any size and/or shape, in preferred embodiments, the solid support is a planar surface. In other embodiments, the solid support can be a fiber optic bundle. In still other embodiments, the solid support comprises one or more microspheres. Microspheres can be of any shape, size or construction. For example, the microsphere can be subnanometer to millimeter size; isometrical to elongated; with or without surface features; or solid or porous. Microsphere may or may not be associated with another surface, such as a planar array, fiber optic substrate or microtiter plate.

In the methods for detecting the presence of a nucleic acid complementary to a capture probe described herein, the capture probe can be one of a plurality of different capture probes. In some of these methods, the capture probes are distributed on the surface of a substrate. For example, the capture probes can be orderly distributed or randomly distributed on the substrate.

In some embodiment of the methods for detecting the presence of a nucleic acid complementary to a capture probe, the step of determining whether the nucleic acid hybridizes with a capture probe can include, but is not limited to, detecting the hybridization by measuring a change in an optical signal.

In addition to the methods described above, also described are hybridization compositions. Such compositions can include a solid support comprising a capture probe and a buffered solution in fluid communication with the capture probe, wherein the buffered solution comprises a double-stranded nucleic acid and an enzyme for converting the double-stranded nucleic acid to a single-stranded nucleic acid. In some embodiments, the double-stranded nucleic comprises DNA.

In some hybridization compositions, the enzyme comprises one or more nucleases. The one or more nucleases can include, but are not limited to, exonuclease III, T7 exonuclease, lambda exonuclease or combinations of such nucleases. In preferred embodiments, the one or more nucleases comprise lambda exonuclease.

In some of the hybridization compositions described herein, the pH of the buffered solution is at least 7.5. In particular embodiments, the pH of the buffered solution is in a range that allows a nuclease to convert a double-stranded nucleic acid to a single-stranded nucleic acid and allows the single stranded nucleic acid to hybridize to a capture probe. In certain embodiments of the hybridization compositions, the buffered solution comprises Tris buffer. In other embodiments, the buffered solution lacks a concentration of monovalent cations sufficient to substantially inhibit the activity of the one or more nucleases. In still other embodiments, the buffered solution essentially lacks monovalent cations. In yet other embodiments, the buffered solution lacks a concentration of phosphate ions sufficient to substantially inhibit the activity of said nuclease. In preferred embodiments, the buffered solution essentially lacks phosphate ions.

Certain preferred hybridization compositions include one or more divalent cations in the buffered solution. In such embodiments, the one or more divalent cations can be present in the buffered solution at a concentration sufficient both to promote activity of the one or more exonucleases used in the conversion step and to permit hybridization of the single-stranded nucleic acid complementary to the capture probe. In some of these embodiments, the one or more divalent cations include, but are not limited to, Mg²⁺, Mn²⁺, Zn²⁺ or combinations thereof. In still other embodiments, the buffered solution can comprise one or more polyamines. In some embodiments, the one or more polyamines can be used in place of, or in addition to, the divalent cations.

Additional preferred hybridization compositions include a solid support comprising a planar surface. Although planar surfaces are preferred in some embodiments, it will be understood that the solid support can be essentially any size and/or shape. For example, the solid support can be a fiber optic bundle. In still other embodiments, the solid support comprises one or more microspheres. Microspheres can be of any shape, size or construction. For example, the microsphere can be subnanometer to millimeter size; isometrical to elongated; with or without surface features; or solid or porous. Microsphere may or may not be associated with another surface, such as a planar array, fiber optic substrate or microtiter plate.

In some embodiments of the hybridization compositions described herein, the capture probe can be one of a plurality of different capture probes. Such capture probes can be distributed on the surface of a substrate in an orderly or random manner.

Other hybridization compositions relate to a solid support comprising a capture probe and a buffered solution that comprises a single-stranded nucleic acid. The buffered solution also comprises one or more divalent cations at a concentration sufficient to permit hybridization between the single-stranded nucleic acid and the capture probe provided that the single-stranded nucleic acid has sufficient complementarity to hybridize with the capture probe. In such embodiments, the one or more divalent cations include, but are not limited to, Mg²⁺, Mn²⁺, Zn²⁺ or combinations thereof. In some embodiments, one or more polyamines can be used in place of, or in addition to, the divalent cations. In such embodiments, the buffered solution can essentially lack monovalent cations. In still other embodiments, the buffered solution can lack a concentration of monovalent cations sufficient to substantially inhibit the activity of the one or more nucleases that were used to convert the double-stranded nucleic acid to a single-stranded nucleic acid. In yet other embodiments, the buffered solution essentially lacks phosphate ions.

### DETAILED DESCRIPTION

Embodiments of the invention relate to methods for hybridizing nucleic acids to capture probes. These methods can include converting double-stranded nucleic acids to single-stranded nucleic acids in a buffered solution, and hybridizing the single-stranded nucleic acids to capture probes in substantially the same or similar buffered solution. In preferred embodiments, both the conversion and the hybridization reactions can occur in a single buffered solution.

In addition to hybridization methods, other embodiments described herein relate to methods for detecting the presence of a nucleic acid complementary to a capture probe. These methods also include converting double-stranded nucleic acids to single-stranded nucleic acids in a buffered solution. The buffered solution comprising the single-stranded nucleic acids is then provided to capture probes and hybridization of the single-stranded nucleic acids with the capture probes is determined. In preferred embodiments, neither the volume of the buffered solution nor the concentration of ions or other charged molecules in the buffered solution is substantially or significantly changed between the conversion and hybridization steps. For example, the pH can remain substantially unchanged between conversion and hybridization steps.

Hybridization compositions are also contemplated herein. These hybridization compositions include a solid support comprising capture probes, and a buffered solution in fluid communication with the capture probes. Some such compositions also include double-stranded nucleic acids and an enzyme for converting the double stranded nucleic acids to single-stranded nucleic acids. Other hybridization compositions described herein include single-stranded nucleic acids and a buffered solution comprising one or more divalent ions at a concentration sufficient to permit hybridization of the single-stranded nucleic acids with capture probes having sufficient complementarity for hybridization.

The remaining description that follows illustrates exemplary embodiments of the subject matter disclosed herein. Those of skill in the art will recognize that there are numerous variations and modifications of the subject matter provided herein that are encompassed by its scope. Accordingly, the description of a certain exemplary embodiments should not be deemed to limit the scope of the present invention.

### Converting Double-Stranded Nucleic Acids to Single-Stranded Nucleic Acids

Some embodiments of the present invention relate to converting double-stranded nucleic acids to single-stranded nucleic acids. As used herein, “nucleic acid” includes both DNA and RNA. In some embodiments, the term “nucleic acid” includes DNA and RNA comprising one or more modified nucleobases or nucleobase analogs. Modified nucleic acids are nucleic acids having nucleotides or structures which may or may not occur in nature. For example, methylation of DNA bases are modifications that often occur in nature, whereas aminations of nucleobases typically do not. Double-stranded nucleic acids can include double-stranded DNA, double-stranded RNA and double-stranded DNA/RNA hybrid molecules. Double-stranded nucleic acids can be denatured or converted to single-stranded nucleic acids by a variety of methods. These methods can include chemical methods, for example, by the addition of chaotropic agents, such as urea, to induce double-stranded nucleic acids to separate into single-stranded molecules. Other methods include physical means, such as heating to a temperature sufficient to disrupt the hydrogen bonding between the two strands of the double-stranded nucleic acids. Still other methods include employing one or more enzymes, such as nucleases, to preferentially digest one of the strands of the double-stranded nucleic acid, thereby leaving an undigested single strand. Furthermore, double-stranded nucleic acid can be converted to a single-stranded nucleic acid by treatment with an enzyme or other reagent that degrades one strand. Enzymatic or chemical treatment can occur under conditions that are not sufficient to disrupt the hydrogen bonding between the two strands of the double-stranded nucleic acids when not in the presence of the degrading reagent or enzyme. For example, the temperature can be sufficiently low that the double-stranded nucleic acid remains hybridized absent the degrading reagent or enzyme. Also, the chemical conditions can be such that the hybrid is not substantially disrupted absent the degrading reagent or enzyme.

The methods exemplified herein for converting double-stranded nucleic acids to single-stranded nucleic acids are also applicable to converting a double-stranded nucleic acid region to a region that is single-stranded. Thus, the methods can be used to produce a nucleic acid having a single-stranded region that is of sufficient length to hybridize to a capture probe or other nucleic acid. In other words, a double-stranded nucleic acid region can be retained in a nucleic acid that is converted to have a single-stranded region in a method of the invention. For example, a nuclease can digest a portion of one strand in a double-stranded nucleic acid such that the product has both a double-stranded region and a single-stranded region. Such molecules can be referred to as a partial duplexes.

There are a variety of nucleases that can be used to digest one strand of a double-stranded nucleic acid, so as to form a single-stranded nucleic acid. Examples of such nucleases include, but are not limited to, lambda exonuclease, exonuclease III, and T7 exonuclease.

In preferred embodiments, a double-stranded nucleic acid can be converted to a single-stranded nucleic acid using lambda exonuclease. Lambda exonuclease is a highly processive exodeoxyribonuclease that selectively digests the 5′-phosphorylated strand of double-stranded DNA in a 5′ to 3′ direction. The enzyme exhibits greatly reduced activity on single-stranded DNA and non-phosphorylated DNA, and has no activity at nicks and limited activity at gaps in DNA (Little, J. W., An exonuclease induced by bacteriophage lambda: II, Nature of the enzymatic reaction, J. Biol. Chem., 242, 679-686, 1967; Mitsis, P. G., Kwagh, J. G., Characterization of the interaction of lambda exonuclease with the ends of DNA, Nucleic Acids Res., 27, 3057-3063, 1999).

Additional nucleases for converting a double-stranded nucleic acid to a single-stranded nucleic acid include exonuclease III. Exonuclease III catalyzes the stepwise removal of mononucleotides from 3′-hydroxyl termini of duplex DNA (Rogers, G. S. and Weiss, B. (1980) L. Grossman and K. Moldave (Eds.), Methods Enzymol., 65, pp. 201-211. New York: Academic Press). During each binding event, only a limited number of nucleotides are removed, resulting in coordinated progressive deletions within the population of DNA molecules (Sambrook, J., Fritsch, E. F. and Maniatis, T. (1989) Molecular Cloning: A Laboratory Manual (2nd. Ed.), 5.84-5.85). Although the enzyme also acts at nicks in duplex DNA to produce single-strand gaps, the preferred substrates are blunt or recessed 3′-termini. The enzyme is not active on single-stranded DNA, and thus 3′-protruding termini are resistant to cleavage. The degree of resistance depends on the length of the extension, with extensions 4 bases or longer being essentially resistant to cleavage. Temperature, salt concentration and the ratio of enzyme to DNA can affect enzyme activity, thus reaction conditions can be tailored to specific applications. Exonuclease III may also have RNase H, 3′-phosphatase and AP-endonuclease activities (Rogers, G. S. and Weiss, B. (1980) L. Grossman and K. Moldave (Eds.), Methods Enzymol., 65, pp. 201-211. New York: Academic Press).

Still other nucleases for converting a double-stranded nucleic acid to a single-stranded nucleic acid include T7 exonuclease. T7 Exonuclease acts in the 5′ to 3′ direction, catalyzing the removal of 5′ mononucleotides from duplex DNA. T7 Exonuclease initiates nucleotide removal from the 5′ termini or at gaps and nicks of double-stranded DNA (Kerr, C. and Sadowski, P. D. (1972) J. Biol. Chem., 247, 305-318). It will degrade both 5′ phosphorylated or 5′ dephosphorylated DNA. The enzyme may also degrade RNA and DNA from RNA/DNA hybrids in the 5′ to 3′ direction (Shinozaki, K. and Okazaki, T. (1978) Nucl. Acids Res., 5, 4245-4261).

Nucleases that specifically recognize RNA/DNA hybrids can also be used to promote strand conversion. For example, RNase H is a nuclease that specifically recognizes RNA/DNA hybrids and specifically degrades the RNA. Because RNase H does not degrade DNA it can be used to convert double-stranded DNA/RNA hybrids to single-stranded DNA molecules. RNase H is often used to destroy the RNA template after first-strand cDNA synthesis and in nuclease protection assays. RNase H can also be used to degrade specific RNA strands when a DNA oligonucleotide is hybridized, such as in the removal of the poly(A) tail from mRNA hybridized to oligo(dT) or the destruction of specific RNA molecules inside or outside the living cell.

### Buffered Solutions

Some embodiments of the methods and compositions described herein employ buffered solutions. In such embodiments, a buffered solution can permit a variety of reactions to occur, for example, conversion of a double-stranded nucleic acid to a single-stranded nucleic acid using a nuclease; hybridization between a single-stranded nucleic acid and a capture probe; and detection of a single-stranded nucleic acid hybridized to a capture probe. Buffered solutions useful in one process, however, may not be useful in other processes. This often presents the problem of having to change from one buffered solution to a different buffered solution between the steps of a multistep process. Neglecting to change the buffered solution to the appropriate solution prior to performing the next step often leads to complete failure of the multistep process. Such is true for the multistep process of exonucleolytically converting double-stranded nucleic acids to single-stranded nucleic acids followed by the step of nucleic acid hybridization. Prior to this invention, it was required that that the first buffered solution used for exonucleolytic conversion be changed to a second different buffered solution for hybridization of the single-stranded nucleic acids with binding partners, such as capture probes. When hybridization is performed using a microarray, changing buffer is troublesome since the volume of buffered solution that is used is extremely small and one cannot easily take advantage of diluting a small volume of the first buffered solution (exonuclease digestion solution) into a large volume of the second buffered solution (hybridization solution). Although there has long been a need for methods to simplify the buffer changing step, there have been no solutions that are efficient and easy to apply.

Disclosed herein are buffered solutions and reaction condition that can be used to mediate both the step of exonucleolytic conversion of a double-stranded nucleic acid to a single-stranded nucleic acid as well as the subsequent hybridization of the single-stranded nucleic acid to a second nucleic acid molecule, such as a capture probe. Surprisingly, the inventors have found the phosphate ions and monovalent cations have an inhibitory effect on exonuclease activity. Furthermore, and quite unexpectedly, the inventors have found that divalent cations as well as other positively charged polyvalent molecules, when used at reasonable concentrations, can provide an environment that permits nucleic acid hybridization. Building on these findings, the inventors have developed buffered solutions and reaction conditions suitable for both the nucleic acid strand conversion and hybridization reactions.

Some embodiments of invention disclosed herein permit the end user to save time and reagents by performing multiple reactions in a single buffered solution. Performing multiple reactions in a single buffered solution is efficient, for example, because there is no need to precipitate and resuspend the product of each reaction before proceeding to another reaction, and in some circumstances reactions can occur simultaneously. Multiple reactions can be carried out in a single reaction vessel, for example, multiple reactions that occur simultaneously. Furthermore, multiple reactions can be carried out sequentially in the same reaction vessel. Reagents can be, but need not be, added to a reaction vessel during the course of multiple reactions

In some embodiments of the present invention, a variety of reactions can occur in substantially the same or similar buffered solution. In such embodiments, substantially the same buffered solution refers to a reaction solution in which a series of reactions can occur, for example, any/all of the aforementioned conversion, hybridization and detection reactions. In some embodiments, the second reaction may take place in a solution that is identical to the first in volume as well as the concentration of certain components. In other embodiments, the concentration of certain reaction components and the volume of the buffered solution used in the second reaction can vary from the concentration of certain reaction components and the volume of the buffered solution used in the first reaction. The variation may be insubstantial, for example, less than 25%, more preferably less than 15%, even more preferably less than 5%, thereby resulting in substantially the same buffer solutions. In other embodiments, the concentration of certain reaction components and the volume of the buffered solution used in the second reaction can vary considerably from the concentration of certain reaction components and the volume of the buffered solution used in the first reaction. The variation can be, for example, more than 25%, more than 50%, more than 75% or even more than 100%.

While a series of reactions can occur in single buffered solution, in some embodiments, the volume or other components of the buffered solution can change, either by removal, addition or as a series of reactions occurs. For example, as reagents for subsequent reactions are added, and/or components of previous reactions are diluted in the reaction volume. In some such embodiments, the buffered solution used for the first reaction might not be the same or substantially the same as the buffered solution used for the second reaction. In these embodiments, however, the buffered solution used for the first reaction can be similar to, or substantially similar to, the buffered solution used for the second reaction.

A variety of buffered solutions can be used with the methods and compositions described herein. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, Tricine, HEPES, TES, MOPS, MOPSO and PIPES. Additionally, other buffer agents that can be used in enzyme reactions, hybridization reactions, and detection reactions are well known in the art. In preferred embodiments, the buffered solution can include Tris.

With respect to the embodiments described herein, the pH of the buffered solution can be modulated to permit any of the described reactions. In some embodiments, the buffered solution can have a pH greater than pH 2.0, greater than pH 2.5, greater than pH 3.0, greater than pH 3.5, greater than pH 4.0, greater than pH 4.5, greater than pH 5.0, greater than pH 5.5, greater than pH 6.0, greater than pH 6.5, greater than pH 7.0, greater than pH 7.5, greater than pH 8.0, greater than pH 8.5, greater than pH 9.0, greater than pH 9.5, greater than pH 10, greater than pH 10.5, greater than pH 11.0, greater than pH 11.5 or greater than pH 12.0. Additionally or alternatively the pH can be less than 12.0, less than 11.5, less than 11.0, less than 10.5, less than 10.0, less than 9.5, less than 9.0, less than 8.5, less than 8.0, less than 7.5, less than 7.0, less than 6.5, less than 6.0, less than 5.5, less than 5.0, less than 4.5, less than 4.0, less than 3.5, less than 3.0, or less than 2.5. In other embodiments, the buffered solution can have a pH ranging, for example, from about pH 2 to about pH 12, from about pH 4 to about pH 10, from about pH 5 to about pH 9, from about pH 6 to about pH 9 or from about pH 7 to about pH 9.

Although not necessarily preferred, in some embodiments, the buffered solution can comprise monovalent cations. Examples of monovalent cations can include, but are not limited to, Li⁺, Na⁺, K⁺ or any other ions of the alkali metals. In preferred embodiments, the buffered solution can comprise monovalent cations at a concentration sufficiently low that the activity of an enzyme used to convert a double-stranded nucleic acid to a single-stranded nucleic acid is not substantially inhibited. In some embodiments, a concentration sufficiently low that the activity of an enzyme used to convert a double-stranded nucleic acid to a single-stranded nucleic acid is not substantially inhibited can be less than about 200 mM, less than about 100 mM, less than about 75 mM, less than about 50 mM, less than about 25 mM, less than about 10 mM, less than about 5 mM, less than about 2 mM, less than about 1 mM, less than about 500 μM, less than about 400 μM, less than about 300 μM, less than about 200 μM, less than about 100 μM, less than about 75 μM, less than about 50 μM, less than about 25 μM, less than about 10 μM, less than about 5 μM, less than about 2 μM, less than about 1 μM, less than about 500 nM, less than about 400 nM, less than about 300 nM, less than about 200 nM, less than about 100 nM, less than about 75 nM, less than about 50 nM, less than about 25 nM, less than about 10 nM, less than about 5 nM, less than about 2 nM, or less than about 1 nM In other embodiments, the buffered solution can contain essentially no monovalent cations or lack monovalent cations.

Although not necessarily preferred, in some embodiments, the buffered solution can comprise phosphate ions. In preferred embodiments, the buffered solution can comprise phosphate ions at a concentration sufficiently low that the activity of an enzyme used to convert a double-stranded nucleic acid to a single-stranded nucleic acid is not substantially inhibited. In some embodiments, a concentration sufficiently low that the activity of an enzyme used to convert a double-stranded nucleic acid to a single-stranded nucleic acid is not substantially inhibited can be less than about 200 mM, less than about 100 mM, less than about 75 mM, less than about 50 mM, less than about 25 mM, less than about 10 mM, less than about 5 mM, less than about 2 mM, less than about 1 mM, less than about 500 μM, less than about 400 μM, less than about 300 μM, less than about 200 μM, less than about 100 μM, less than about 75 μM, less than about 50 μM, less than about 25 μM, less than about 10 μM, less than about 5 μM, less than about 2 μM, less than about 1 μM, less than about 500 nM, less than about 400 nM, less than about 300 nM, less than about 200 nM, less than about 100 nM, less than about 75 nM, less than about 50 nM, less than about 25 nM, less than about 10 nM, less than about 5 nM, less than about 2 nM, or less than about 1 nM In other embodiments, the buffered solution can contain essentially no phosphate ions or lack phosphate ions.

In preferred embodiments, the buffered solution can comprise one or more divalent cations. Examples of divalent cations can include, but are not limited to, Mg²⁺, Mn²⁺, Zn²⁺ and Ca²⁺. In preferred embodiments, the buffered solution can contain one or more divalent cations at a concentration sufficient to permit hybridization of a single-stranded nucleic acid complementary to a capture probe. In some embodiments, a concentration sufficient to permit hybridization of a single-stranded nucleic acid complementary to a capture probe can be more than about 1 μM, more than about 2 μM, more than about 5 μM, more than about 10 μM, more than about 25 μM, more than about 50 μM, more than about 75 μM, more than about 100 μM, more than about 200 μM, more than about 300 μM, more than about 400 μM, more than about 500 μM, more than about 750 μM, more than about 1 mM, more than about 2 mM, more than about 5 mM, more than about 10 mM, more than about 20 mM, more than about 30 mM, more than about 40 mM, more than about 50 mM, more than about 60 mM, more than about 70 mM, more than about 80 mM, more than about 90 mM, more than about 100 mM, more than about 150 mM, more than about 200 mM, more than about 250 mM, more than about 300 mM, more than about 350 mM, more than about 400 mM, more than about 450 mM, more than about 500 mM, more than about 550 mM, more than about 600 mM, more than about 650 mM, more than about 700 mM, more than about 750 mM, more than about 800 mM, more than about 850 mM, more than about 900 mM, more than about 950 mM or more than about 1M.

In some embodiments, the buffered solution can comprise one or more polyamines. Examples of polyamines include, but are not limited to, spermine and spermidine. In preferred embodiments, a buffered solution can comprise one or more polyamines at a concentration at a concentration sufficient to permit hybridization of a single-stranded nucleic acid complementary to a capture probe. In some embodiments, a concentration sufficient to permit hybridization of a single-stranded nucleic acid complementary to a capture probe can be more than about 1 μM, more than about 2 μM, more than about 5 μM, more than about 10 μM, more than about 25 μM, more than about 50 μM, more than about 75 μM, more than about 100 μM, more than about 200 μM, more than about 300 μM, more than about 400 μM, more than about 500 μM, more than about 750 μM, more than about 1 mM, more than about 2 mM, more than about 5 mM, more than about 10 mM, more than about 20 mM, more than about 30 mM, more than about 40 mM, more than about 50 mM, more than about 60 mM, more than about 70 mM, more than about 80 mM, more than about 90 mM, more than about 100 mM, more than about 150 mM, more than about 200 mM, more than about 250 mM, more than about 300 mM, more than about 350 mM, more than about 400 mM, more than about 450 mM, more than about 500 mM, more than about 550 mM, more than about 600 mM, more than about 650 mM, more than about 700 mM, more than about 750 mM, more than about 800 mM, more than about 850 mM, more than about 900 mM, more than about 950 mM or more than about 1M. In other embodiments, the buffered solution can comprise both one or more divalent cations and one or more polyamines.

In a preferred embodiment, the buffered solution comprises one or more divalent cations and/or one or more polyamines and lacks monovalent cations and phosphate ions.

### Hybridization

Some embodiments of the present invention relate to hybridization between single-stranded nucleic acids and capture probes. As described further herein, capture probes can be short nucleic acids or oligonucleotides. Short nucleic acids typically have a length of 1000 nucleotide or less. Other embodiments of the present invention relate to hybridization between single-stranded nucleic acids and other nucleic acid molecules having a length greater than 1000 base pairs. Several useful properties of single-stranded nucleic acids are exemplified below. It will be understood that a single-stranded region of a nucleic acid can have similar useful properties even if the nucleic acid also has a double-stranded region.

Hybridization occurs when hydrogen bonds form between complementary nucleotide bases, for example, T-A, C-G, and A-U. Complementary nucleic acids comprise complementary bases with the capacity for precise pairing between two nucleotides, for example, if a nucleotide at a certain position in the sequence of nucleotides of an single-stranded nucleic acid is capable of hydrogen bonding with a nucleotide at the same position in the sequence of nucleotides of a capture probe, then the single-stranded nucleic acid and capture probe are considered to be complementary to each other at that position. The single-stranded nucleic acid and the capture probe are complementary to each other when a sufficient number of corresponding positions in each molecule are occupied by nucleotides which can hydrogen bond with each other. Accordingly, complementary does not necessarily mean that two hybridizing nucleic acid stranded have 100% nucleotide complementarity in the hybridizing region. For example, in some embodiments, hybridizing nucleic acids can have less than 100% complementarity, less than 99% complementarity, less than 98% complementarity, less than 97% complementarity, less than 96% complementarity, less than 95% complementarity, less than 94% complementarity, less than 93% complementarity, less than 92% complementarity, less than 91% complementarity, less than 90% complementarity, less than 89% complementarity, less than 88% complementarity, less than 87% complementarity, less than 86% complementarity, less than 85% complementarity, less than 84% complementarity, less than 83% complementarity, less than 82% complementarity, less than 81% complementarity, less than 80% complementarity, 79% complementarity, less than 78% complementarity, less than 77% complementarity, less than 76% complementarity, less than 75% complementarity, less than 74% complementarity, less than 73% complementarity, less than 72% complementarity, less than 71% complementarity or less than 70% complementarity in the hybridizing region provided that the complementarity is sufficient to promote hybridization under the conditions used. In preferred embodiments, the hybridization occurs between specific complementary sequences and not between non-complementary sequences.

The ability of a single-stranded nucleic acid and a capture probe to hybridize to one another can be affected by the number of complementary nucleotides and the relative positions of those complementary nucleotides in the single-stranded nucleic acid and capture probe. For example, a single-stranded nucleic acid containing a greater number of complementary nucleotides in a contiguous sequence can have a higher degree of complementarity than a single-stranded nucleic acid contains a lower number of complementary nucleotides with non-complementary nucleotides dispersed therein. In addition, as indicated above, the ability of a single-stranded nucleic acid and capture probe to hybridize to one another can be modulated by varying the conditions in which the hybridization occurs.

In some embodiments of the methods and compositions described herein, a single-stranded nucleic acid can contain at least one sequence that can hybridize to a sequence contained in a capture probe. Such sequences that can hybridize include complementary nucleotides. In certain embodiments, a sequence that can hybridize can contain a contiguous sequence of complementary nucleotides. For example, a single-stranded nucleic acid can contain at least one contiguous sequence complementary to at least one sequence in capture probe. In such embodiments, the at least one contiguous sequence of complementary nucleotides contained in the capture probe and/or single-stranded nucleic acid can have a length of at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotide, at least 34 nucleotides, at least 35 nucleotides, at least 36 nucleotides, at least 37 nucleotides, at least 38 nucleotides, at least 39 nucleotides, at least 40 nucleotides, at least 41 nucleotides, at least 42 nucleotides, at least 43 nucleotides, at least 44 nucleotides, at least 45 nucleotides, at least 46 nucleotides, at least 47 nucleotides, at least 48 nucleotides, at least 49 nucleotides, at least 50 nucleotides, at least 51 nucleotides, at least 52 nucleotides, at least 53 nucleotides, at least 54 nucleotides, at least 55 nucleotides, at least 56 nucleotides, at least 57 nucleotides, at least 58 nucleotides, at least 59 nucleotides, at least 60 nucleotides, at least 61 nucleotides, at least 62 nucleotides, at least 63 nucleotides, at least 64 nucleotides, at least 65 nucleotides, at least 66 nucleotides, at least 67 nucleotides, at least 68 nucleotides, at least 69 nucleotides, at least 70 nucleotides, at least 71 nucleotides, at least 72 nucleotides, at least 73 nucleotides, at least 74 nucleotides or at least 75 nucleotides.

In other embodiments, the sequence that can hybridize to another sequence can contain non-complementary nucleotides. In such embodiments, a sequence that can hybridize can contain 1 non-complementary nucleotide, 2 non-complementary nucleotides, 3 non-complementary nucleotides, 4 non-complementary nucleotides, 5 non-complementary nucleotides, 6 non-complementary nucleotides, 7 non-complementary nucleotides, 8 non-complementary nucleotides, 9 non-complementary nucleotides, 10 non-complementary nucleotides, 11 non-complementary nucleotides, 12 non-complementary nucleotides, 13 non-complementary nucleotides, 14 non-complementary nucleotides, 15 non-complementary nucleotides, 16 non-complementary nucleotides, 17 non-complementary nucleotides, 18 non-complementary nucleotides, 19 non-complementary nucleotides, 20 non-complementary nucleotides, 25 non-complementary nucleotides, 30 non-complementary nucleotides, 35 non-complementary nucleotides, 40 non-complementary nucleotides, 45 non-complementary nucleotides, or 50 non-complementary nucleotides.

As is known in the art, the ability of a single-stranded nucleic acid and capture probe to hybridize to one another can be modulated by varying the conditions in which the hybridization occurs. Such conditions are well known in the art and can include, for example, pH, temperature, concentration of salts, and the presence of particular molecules in the hybridization reaction. Under conditions of low stringency, a capture probe and single-stranded nucleic acid with a low degree of complementarity may be able to hybridize to one another. Conversely, under more highly stringent conditions, only capture probes and single-stranded nucleic acids with a high degree of complementarity are likely to hybridize to one another.

In certain embodiments, hybridization of the single-stranded nucleic acid and capture probe can be made to occur under conditions with high stringency. One condition that greatly affects stringency is temperature. In general, increasing the temperature at which the hybridization is performed increases the stringency. As such, the hybridization reactions described herein can be performed at a different temperature depending on the desired stringency of hybridization. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can further altered by the addition or removal of components of the buffered solution.

In particular embodiments, a probe can be resistant to exonuclease degradation. For example the probe can have a non natural backbone that can not be cleaved by a particular exonuclease such as a protein nucleic acid backbone. A probe can include a blocking group that prevents or inhibits exonuclease degradation. For example, a blocking group can present at the 3′ end of a probe or at the 5′ end of the probe. A blocking group at the 3′ end can prevent degradation of the probe by exonuclease III. A blocking group at the 5′ end can prevent degradation of the probe by lambda exonuclease or T7 exonuclease.

### Arrays

Some embodiments of the methods and compositions described herein employ arrays. In some embodiments, an array refers to a solid support comprising a plurality of capture probes at spatially distinguishable locations. Arrays can have one or more surfaces on which capture probes are distributed. In some embodiments, all of the capture probes distributed on an array surface are identical to each other. In other embodiments, some of the capture probes distributed on the array surface are identical to each other but different from one or more other capture probes distributed on the array surface. In still other embodiments, most or all of the capture probes distributed on an array surface are different from each other.

In embodiments where capture probes are distributed on an array surface, the capture probes can be distributed at discrete sites. In some embodiments, a discrete site is a feature having a plurality of copies of a particular capture probe. Thus, an array can comprise a plurality of discrete sites or features. In some embodiments, a space separates each discrete site from another such that the discrete sites are noncontiguous. In other embodiments, the discrete sites are contiguous. For some of the arrays described herein, discrete sites can be present on the array surface at a density of greater than 10 discrete sites per square millimeter. For other arrays, discrete sites can be present on the array surface at a density of greater than 100 discrete sites per square millimeter, greater than 1000 discrete sites per square millimeter, greater than 10,000 discrete sites per square millimeter, greater than 100,000 discrete sites per square millimeter, greater than 1,000,000 discrete sites per square millimeter, greater than 10,000,000 discrete sites per square millimeter, greater than 100,000,000 discrete sites per square millimeter or greater than 1,000,000,000 discrete sites per square millimeter.

In some embodiments of the present invention, capture probes refer to molecules that are associated with an array that comprise one or more nucleic acids. In some embodiments, the capture probes can be nucleic acids that bind, hybridize or otherwise interact with one or more single-stranded nucleic acids that are transferred to the array. In preferred embodiments, the capture probes are oligonucleotides or otherwise comprise one or more oligonucleotides. In such embodiments, the capture probes comprise oligonucleotides that have an average length of 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotide, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides. 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides or 75 nucleotides. In other embodiments, oligonucleotides have an average length of greater than 75 nucleotides.

With respect to some of the arrays described herein, the capture probes are coupled to an array surface. Such coupling can be via a direct attachment of the capture probe to the array surface. Direct attachment can include, but is not limited to, covalent attachment, non-covalent attachment, and adsorptive attachment. Alternatively, capture probes can be attached to the array surface via one or more intermediate molecules or particles. A probe can be attached to an array surface via the 3′ end of the probe or via the 5′ end of the probe. The attachment can block or inhibit enzymatic degradation of the probe. For example, attachment of a probe to a surface via the 3′ end can prevent degradation of the probe by exonuclease III. Attachment of a probe to a surface via the 5′ end can prevent degradation of the probe by lambda exonuclease or T7 exonuclease. Exemplary attachments are described, for example, in US Patent Application Publication No. 2006/0127930 A1, which is incorporated herein by reference and also in references listed below in regard to various arrays.

Depending on the deposition method, the capture probes can be distributed on the array surface in either a random or ordered distribution. For example, in some embodiments, capture probes are synthesized directly on the array surface such that the position of each capture probe is known. In such embodiments, the capture probes can be synthesized in any order that is desired. For example, capture probes may be grouped by functionality or binding affinity for a particular molecule. In other embodiments, the capture probes are synthesized then coupled to an array surface. In such embodiments, the capture probes can be coupled to specific areas of the array surface such that the specific areas of the array surface comprise a defined set of capture probes.

With respect to other arrays described herein, capture probes are not attached directly to the array, but rather, they are associated with the array through intermediate structures, such as particles. In such embodiments, a plurality of particles is distributed on the array. The plurality of particles can comprise particles that have one or more capture probes coupled thereto, as well as particles that do not have any capture probes coupled thereto. In some embodiments, all particles of the plurality of particles have one or more identical capture probes coupled thereto. In certain embodiments, where pluralities of particles are used, the capture probes coupled the particles are identical to each other such that all particles have the same identical capture probes coupled thereto. In other embodiments, where pluralities of particles are used, some or all of the capture probes coupled the particles are different from each other such that some particles have capture probes coupled thereto that are different from the capture probes attached to other particles. In preferred embodiments, the particles are inanimate, non-living beads or microspheres. In further embodiments, the microspheres can be porous.

In certain embodiments of the present invention, a plurality of particles is distributed on the surface of an array. In some embodiments, the particles are distributed on the array such that one or more particles end up in a depression present on the array. In some embodiments, the depressions are configured to hold a single particle. In other embodiments, the depressions are configured to hold thousands, or even millions, of particles.

The plurality of particles can be distributed on the array so that they are orderly or randomly distributed. In particular embodiments, an array can comprise a particle-based analytic system in which particles carrying different functionalities are distributed on an array comprising a patterned surface of discrete sites, each capable of binding an individual particle.

Arrays described herein can have a variety of surfaces. In some embodiments, an array surface can comprise a fiber optic bundle. Arrays having planar surfaces or surfaces with one or more depressions, channels or grooves are particularly useful. In addition, some of the arrays have a non-porous surface. In some embodiments, the entire array is non-porous. In other embodiments, the array has at least one porous or semi-porous surface but is primarily non-nonporous.

Preferred array materials include, but are not limited to glass, silicon, plastic or non-reactive polymers. Arrays described herein can be rigid or flexible. In some embodiments, the array is rigid, whereas in other embodiments, the array is not rigid but comprises at least one rigid surface. Other arrays contemplated herein can comprise a flexible array substrate having a flexible support, such as that described in U.S. patent application Ser. No. 10/285,759, now U.S. Pat. No. 7,422,911, the disclosures of which are hereby incorporated expressly by reference in their entireties.

Some of the arrays described herein include one or more patterned surfaces. In some embodiments, the array surface can comprise one or more discrete sites. In certain embodiments, the discrete sites can be depressions, such as wells, grooves, channels or indentations. Depressions can be sized so as to accommodate as few as one particle or as many as several million particles.

In further embodiments an array can comprise a composite array (array of subarrays) as described in U.S. Pat. No. 6,429,027 or U.S. Pat. No. 5,545,531, the disclosures of which are hereby incorporated expressly by reference in their entirety. Composite arrays can comprise a plurality of individual arrays on a surface of the array or distributed in depressions present on the array surface. The plurality of individual arrays on a surface of the array or distributed in depressions present on the array surface can be referred to as subarrays. For example, in a composite array, a single subarray can be present in each of a plurality of depressions present on the array. In other embodiments, multiple subarrays can be present in each depression of a plurality of depressions present on the array. Individual subarrays can be different from each other or can be the same or similar to other subarrays present on the array. Accordingly, in some embodiments, the surface of a composite array can comprise a plurality of different and/or a plurality of identical, or substantially identical, subarrays. Moreover, in some embodiments, the surface of an array comprising a plurality of subarrays can further comprise an inter-subarray surface. By “inter-subarray surface” or “inter-subarray spacing” is meant the portion of the surface of the array not occupied by subarrays. In some embodiments, “inter-subarray surface” refers to the area of array surface between a first subarray and an adjacent second subarray.

Subarrays can include some or all of the features of the arrays described herein. For example, subarrays can include depressions that are configured to contain one or more particles. Moreover, subarrays can further comprise their own subarrays.

Exemplary arrays that can be utilized in combination with the methods and compositions described herein include, without limitation, those in which beads are associated with a solid support, examples of which are described in U.S. Pat. No. 6,355,431; U.S. Pat. No. 6,327,410; U.S. Pat. No. 6,770,441; US Published Patent Application No. 2004/0185483; US Published Patent Application No. 2002/0102578 and PCT Publication No. WO 00/63437, each of which is incorporated herein by reference in its entirety. Beads can be located at discrete locations, such as wells, on a solid-phase support, whereby each location accommodates a single bead.

Any of a variety of other arrays known in the art or methods for fabricating such arrays can be used. Commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752 and 6,482,591, each of which is hereby incorporated by reference in its entirety. A spotted microarray can also be used in a method of the invention. An exemplary spotted microarray is a CodeLink™ Array available from Amersham Biosciences. Another microarray that is useful in the invention is one that is manufactured using inkjet printing methods such as SurePrint™ Technology available from Agilent Technologies.

In a particular embodiment, clustered arrays of nucleic acid colonies can be prepared as described in U.S. Pat. No. 7,115,400; US Published Patent Application No. 2005/0100900 A1; PCT Publication No. WO 00/18957 or PCT Publication No. WO 98/44151 (the contents of which are herein incorporated by reference in their entireties). Such methods are known as bridge amplification or solid-phase amplification and are particularly useful for sequencing applications.

### Methods of Detecting Nucleic Acids

Some embodiments of the methods and compositions disclosed herein relate to determining whether a single-stranded nucleic acid hybridizes to a capture probe. In some embodiments, a binding reaction can be detected between a single-stranded nucleic acid and one or more capture probes on the surface of an array. In preferred embodiments, the binding of at least 100 different nucleic acids can be detected. In more preferred embodiments, the binding of at least 1,000,000 different nucleic acids can be detected.

In some embodiments, the binding reaction between a single stranded nucleic acid and a capture probe can indicate the presence in a sample of a nucleic acid complementary to the capture probe.

In some embodiments, a binding reaction can be detected by a variety of methods, such as by determining the change in a signal. In certain embodiments, the hybridization between a single-stranded nucleic acid and a capture probe can be determined by measuring a change in an optical signal. For example, in some embodiments a sample comprising one or more nucleic acids can be provided to an array. One or more target nucleic acids in the sample can be detected by determining a change in a signal upon hybridization of the target nucleic acid or by adding one or more molecules that produce a signal when the target nucleic acid is bound to a capture probe but which do not produce a signal when no target nucleic acid is bound. As such, in some embodiments, the detection methods described herein can be used to determine the presence or absence of one or more nucleic acids in a sample.

In other embodiments, the detection methods described herein can be used to determine the nature or composition of an unknown substance or mixture. In some such embodiments, the detection methods described herein can be used to detect the presence of one or more nucleic acids or nucleic acid variants in a sample. In some embodiments, the sample can be obtained from an organism, such as a plant, bacterium, or mammal such as a human. In some such embodiments, the sample contains all or a portion of the genomic DNA of the organism or derivatives of the genomic DNA, including, but not limited to, mRNA, gDNA copies or adapter-linked gDNA copies and derivatives. The sample can be isolated from an organism. Alternatively or additionally, the sample can include a product of an amplification reaction performed using nucleic acid template from an organism. Exemplary amplification methods include, but are not limited to, polymerase chain reaction (PCR), which is described, for example, in U.S. Pat. No. 4,683,195 and U.S. Pat. No. 4,683,202; rolling circle amplification (RCA), which is described, for example, in U.S. Pat. No. 6,344,329 and U.S. Pat. No. 6,593,086; ligation chain reaction (LCR), which is described, for example, in U.S. Pat. No. 5,185,243, U.S. Pat. No. 5,679,524 and U.S. Pat. No. 5,573,907; and other amplification methods known in the art such as those described in U.S. Pat. No. 6,355,431 B1, US Patent Application Publication No. 2003/0211489 A1 and US Patent Application Publication No. 2005/0181394 A1. Each of the foregoing references is incorporated herein by reference. In other embodiments, the sample can contain synthetic nucleic acids, which may or may not correspond to one or more nucleic acids present in one or more organisms.

In some embodiments, a sample comprising nucleic acids from one or more sources can be provided to the array. In such embodiments, the capture probes on the array function as hybridization probes that bind to the nucleic acid sample applied to the array. The binding of a nucleic acid at any particular position can be detected by determining a change in a signal. Such methods are well known in the art. In other embodiments, the capture probes can function as primers permitting the priming of a nucleotide synthesis reaction using a nucleic acid from the nucleic acid sample as a template. In this way, information regarding the sequence of the nucleic acids supplied to the array can be obtained. In some embodiments, nucleic acids hybridized to capture probes on the array can serve as sequencing templates if primers that hybridize to the nucleic acids bound to the capture probes and sequencing reagents are further supplied to the array. Methods of sequencing using arrays have been described previously in the art.

As described above, one or more sequencing steps can be performed subsequent to the conversion and hybridization steps. Such sequencing steps can include, but are not limited to, sequencing-by-synthesis (SBS). In SBS, four fluorescently labeled modified nucleotides are used to determine the sequence of nucleotides for nucleic acids present on the surface of a support structure such as a flowcell. Exemplary SBS systems and methods which can be utilized with the apparatus and methods set forth herein are described in US Patent Application Publication No. 2007/0166705, US Patent Application Publication No. 2006/0188901, U.S. Pat. No. 7,057,026 US Patent Application Publication No. 2006/0240439, US Patent Application Publication No. 2006/0281109, PCT Publication No. WO 05/065814, US Patent Application Publication No. 2005/0100900, PCT Publication No. WO 06/064199 and PCT Publication No. WO 07/010,251, each of which is incorporated herein by reference in its entirety.

In other uses of the methods herein and compositions described herein, arrayed nucleic acids are treated by several repeated cycles of an overall sequencing process. The nucleic acids are prepared such that they include an oligonucleotide primer (capture probe) hybridized to an unknown target sequence (single-stranded nucleic acid). To initiate the first SBS sequencing cycle, one or more differently labeled nucleotides and a DNA polymerase can be introduced to the array. Either a single nucleotide can be added at a time, or the nucleotides used in the sequencing procedure can be specially designed to possess a reversible termination property, thus allowing each cycle of the sequencing reaction to occur simultaneously in the presence of all four labeled nucleotides (A, C, T, G). Following nucleotide addition, the features on the surface can be detected to determine the identity of the incorporated nucleotide (based on the labels on the nucleotides). Then reagents can be added to remove the blocked 3′ terminus (if appropriate) and to remove labels from each incorporated base. Reagents, enzymes and other substances can be removed between steps by washing. Such cycles are then repeated and the sequence of each cluster is read over the multiple chemistry cycles.

Other sequencing methods that use cyclic reactions can be used, such as those wherein each cycle can include steps of delivering one or more reagents to nucleic acids, for example, pyrosequencing and sequencing-by-ligation. Useful pyrosequencing reactions are described, for example, in US Patent Application Publication No. 2005/0191698 and U.S. Pat. No. 7,244,559, each of which is incorporated herein by reference. Sequencing-by-ligation reactions are described, for example, in Shendure et al. Science 309:1728-1732 (2005); U.S. Pat. No. 5,599,675; and U.S. Pat. No. 5,750,341, each of which is incorporated herein by reference in its entirety.

Double-stranded nucleic acid products of other assays can be converted to single-stranded nucleic acids and hybridized using methods set forth herein. Exemplary assays include, those utilizing polymerase chain reaction (PCR), oligonucleotide ligation assay (OLA), extension ligation and combinations or variants thereof. OLA involves the template-dependent ligation of two smaller probes into a single long probe, using a target sequence as the template. In a particular embodiment, a single-stranded target sequence includes a first target domain and a second target domain, which are adjacent and contiguous. A first OLA probe and a second OLA probe can be hybridized to complementary sequences of the respective target domains. The two OLA probes are then covalently attached to each other to form a modified probe. In embodiments where the probes hybridize directly adjacent to each other, covalent linkage can occur via a ligase. A ligated product produced in an OLA reaction can be treated to remove the template and hybridize the ligated product using methods set forth herein. In particular embodiments, the ligated product can be subsequently amplified by a PCR reaction and the PCR product converted to a single nucleic acid for hybridization to a probe.

Alternatively, an extension ligation assay (such as the GoldenGate™ assay available from Illumina Inc.) can be used wherein a pair of probes are hybridized to a template strand at non-contiguous positions and one or more nucleotides are added along with one or more agents that join the probes via the added nucleotides. Exemplary agents include, for example, polymerases and ligases. The joined probes can be, but need not be, subjected to a PCR amplification reaction. In embodiments in which ligation products are PCR amplified, the ligation probes can include primer regions. For example, a population of different ligation probe pairs can include members that have tails containing the same set of priming site sequences such that a pair of universal primers can be used for PCR amplification. Further conditions for OLA, extension ligation, and/or PCR assays, as well as other assays that are useful in combination with the methods set forth herein are described, for example, in U.S. Pat. No. 6,355,431 B1, US Patent Application Publication No. 2003/0211489 A1 and US Patent Application Publication No. 2005/0181394 A1, each of which is incorporated herein by reference.

In embodiments wherein random arrays are used, one or more single-stranded molecules can be provided to the array using the methods described herein. Methods of decoding random arrays are described in, for example, U.S. Pat. No. 7,060,431, the disclosure of which is incorporated herein by reference in its entirety. In brief, a decoding allows one to determine the position and identity of specified capture probes on random arrays. This is particularly useful when a mixture of target molecules are supplied to the array together at substantially the same time because it provides a means to determine the identity of the target molecules present in the sample.

### Hybridization Compositions

Some embodiments of the present invention relate to hybridization compositions. Such hybridization compositions can include any or all of the following components: a buffered solution, a nucleic acid, a nuclease, a capture probe, and an array. The nucleic acid can include double-stranded nucleic acid, and/or single-stranded nucleic acid. For example, in a preferred embodiment, the hybridization composition comprises a solid support comprising a capture probe and a buffered solution in fluid communication with the capture probe. The buffered solution comprises a double-stranded nucleic acid and an enzyme for converting the double-stranded nucleic acid to a single-stranded nucleic acid. In an especially preferred embodiment, the enzyme comprises one or more exonucleases that are capable of converting the double-stranded nucleic acid to a single-stranded nucleic acid, such as lambda exonuclease.

In another preferred embodiment, the hybridization composition comprises a single-stranded nucleic acid rather than a double-stranded nucleic acid. In such compositions, the conversion enzyme is not necessarily provided. Accordingly, such hybridization compositions relate to a solid support comprising a capture probe and a buffered solution comprising a single-stranded nucleic acid. The buffered solution also comprises one or more divalent cations at a concentration sufficient to permit hybridization of the capture probe with the single-stranded nucleic acid provided that the capture probe and the single-stranded nucleic acid have sufficient complementarity to hybridize under the desired stringency conditions. For example, if under high stringency conditions 100% or near 100% complementarity may be necessary to facilitate hybridization under high stringency conditions, such as high temperatures. In such embodiments, the divalent cations include, but are not limited to, Mg²⁺, Mn²⁺, Zn²⁺ or combinations thereof. In some embodiments, one or more polyamines can be used in place of, or in addition to, the divalent cations. In such embodiments, the buffered solution can essentially lack monovalent cations. In still other embodiments, the buffered solution can lack a concentration of monovalent cations sufficient to substantially inhibit the activity of the one or more nucleases that were used to convert the double-stranded nucleic acid to a single-stranded nucleic acid. In yet other embodiments, the buffered solutions can essentially lack phosphate ions.

In some embodiments of the hybridization compositions described herein, the double-stranded nucleic acid comprises a DNA duplex, an RNA duplex and/or a DNA/RNA duplex.

In preferred embodiments of the hybridization compositions described herein, the pH of the buffered solution is at least 7.5. In other preferred embodiments the pH is greater than 7.5. In more preferred embodiments, the buffered solution comprises Tris buffer. However, as discussed above, in some embodiments, the pH of the buffered solution can range from pH 2.0 to pH 11.0. Also as described above, the buffered solution can lack a concentration of monovalent cations and/or phosphate ions sufficient to substantially inhibit the activity of the one or more nucleases, but preferably the buffered solution essentially lacks monovalent cations and/or phosphate ions.

Certain preferred hybridization compositions include divalent cations in the buffered solution. Examples of divalent cations can include, but are not limited to, Mg²⁺, Mn²⁺, Zn²⁺ and Ca²⁺. In preferred embodiments, the buffered solution can contain one or more divalent cations at a concentration sufficient to permit hybridization of a single-stranded nucleic acid complementary to a capture probe. In some embodiments, a concentration sufficient to permit hybridization of a single-stranded nucleic acid complementary to a capture probe can be more than about 1 μM, more than about 2 μM, more than about 5 μM, more than about 10 μM, more than about 25 μM, more than about 50 μM, more than about 75 μM, more than about 100 μM, more than about 200 μM, more than about 300 μM, more than about 400 μM, more than about 500 μM, more than about 750 μM, more than about 1 mM, more than about 2 mM, more than about 5 mM, more than about 10 mM, more than about 20 mM, more than about 30 mM, more than about 40 mM, more than about 50 mM, more than about 60 mM, more than about 70 mM, more than about 80 mM, more than about 90 mM, more than about 100 mM, more than about 150 mM, more than about 200 mM, more than about 250 mM, more than about 300 mM, more than about 350 mM, more than about 400 mM, more than about 450 mM, more than about 500 mM, more than about 550 mM, more than about 600 mM, more than about 650 mM, more than about 700 mM, more than about 750 mM, more than about 800 mM, more than about 850 mM, more than about 900 mM, more than about 950 mM or more than about 1M.

Additional preferred hybridization compositions include a solid support comprising a planar surface. Although planar surfaces are preferred in some embodiments, it will be understood that the solid support can be any essentially any size and/or shape. For example, the solid support can be a fiber optic bundle. In still other embodiments, the solid support comprises a microsphere. Microspheres can be of any shape, size or construction. For example, the microsphere can be subnanometer to millimeter size; isometrical to elongated; with or without surface features; or solid or porous. Microsphere may or may not be associated with another surface, such as a planar array, fiber optic substrate or microtiter plate.

In some embodiments of the hybridization compositions described herein, the capture probe can be one of a plurality of different capture probes. Such capture probes can be distributed on the surface of a substrate in an orderly or random manner.

### EXAMPLES

Having generally described embodiments of the present invention, a further understanding can be obtained by reference to certain specific examples which are provided herein for purposes of illustration only, and are not intended to be limiting.

### Example 1

### Single Buffer Nucleic Acid Conversion and Hybridization

This example describes evaluation of divalent cations for use in supporting nucleic acid conversion and hybridization in a single solution. Several divalent cations including magnesium, zinc, manganese, polyamine spermine and polyamine spermadine were evaluated individually and in various combinations.

Sixteen different DNA samples were used to produce PCR products of about 150 basepairs using a Phos-T7 primer. The PCR amplification products were subjected to exonuclease treatment by incubation in 2×MSS for 1 hour, at 45° C., with shaking on a VWR Vortemp at 850 rpm. The 2×MSS contained 0.1 mM Tris pH 8.0, 0.1% S9, 10% polyethylene glycol, 10 mM dithiothreitol, 10% dimethylsulfoxide, 10% sucrose, 48 units lambda exonuclease, 0.002 mg/ml hybloc, 0.075 M NaCl. Divalent cations were added to 2×MSS as described below.

Four separate series of exonuclease treatments were carried out using an equivalent amount of PCR amplification product. For each series one of the following divalent cations was added as the “variable cation”: zinc chloride, manganese chloride, polyamine spermine and polyamine spermidine. Additionally, for some samples of the series, magnesium chloride was also added. Table 1 shows the concentrations for the variable cation and magnesium chloride in separate samples of each series. Following exonuclease treatment, the 8 samples from each series were loaded into individual wells of an Invitrogen Pre-cast E-Gel (4% agarose), separated by electrophoresis and the separated products visualized by ethidium bromide staining. As a control, each gel also included a lane loaded with undigested PCR amplification product in an amount equivalent to the amount introduced into each exonuclease treatment sample.

TABLE 1

| | Millimolar concentration of cation in 2x MSS | | | | | | | |
|---|---|---|---|---|---|---|---|---|
| MgCl₂ | 50 | 0 | 0 | 0 | 10 | 20 | 30 | 40 |
| Variable cation | 0 | 75 | 50 | 25 | 40 | 30 | 20 | 10 |

For each of the four variable cations tested, the gels were evaluated with respect to the size and signal intensity for the band from each sample in the titration series relative to the size and signal intensity for the band in the undigested PCR amplification product. When zinc chloride was used as the variable salt, some precipitation of sample components was observed. Furthermore, this salt was not well tolerated by the exonuclease enzyme. Manganese chloride yielded reduced intensity for the PCR product band for all samples in comparison to the undigested PCR amplification product, thereby indicating that manganese chloride was an acceptable replacement for magnesium chloride. Spermine yielded reduced intensity for the PCR product band for all samples in comparison to the undigested PCR amplification product. Low concentrations of spermine when used in combination with magnesium chloride resulted in an increased efficiency of lambda exonuclease cleavage compared to use of magnesium chloride at similar concentrations, thereby indicating that spermine was an acceptable replacement for magnesium chloride and provides improved results when used in combination with magnesium chloride. Spermidine showed similar results to those observed from spermine indicating that spermidine was an acceptable replacement for magnesium chloride and that spermidine provides improved results when used in combination with magnesium chloride.

In a separate set of experiments, PCR products were obtained as described above and hybridized to capture probes of an Illumina BeadArray in the presence of 2×MSS to which divalent cations had been added as shown in Table 2. The hybridization was allowed to proceed for 1 hour, at 45° C., shaking on a VWR Vortemp at 850 rpm.

TABLE 2

| | Millimolar concentration of cation in 2x MSS | | | | | | | | | | | |
|---|---|---|---|---|---|---|---|---|---|---|---|---|
| MgCl₂ | 50 | | 20 | 30 | 40 | 20 | 30 | 40 | | 20 | 30 | 40 |
| Spermine | | 25 | 30 | 20 | 10 | | | | | | | |
| Spermidine | | | | | | 30 | 20 | 10 | | | | |
| MnCl₂ | | | | | | | | | 50 | 30 | 20 | 10 |

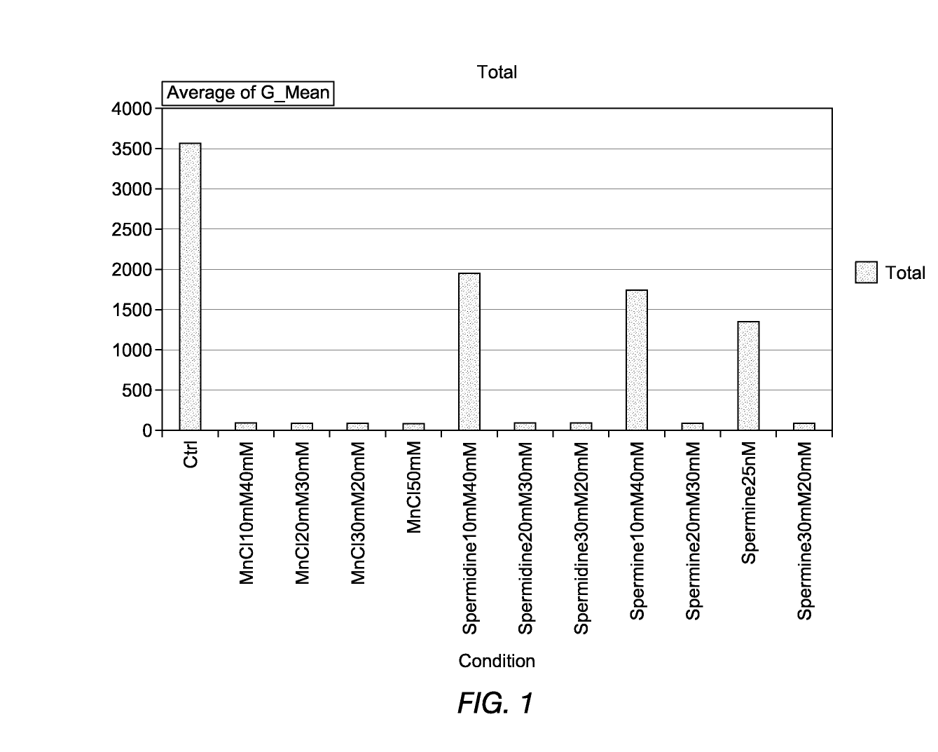

As shown in FIG. 1, hybridization was most efficient in the presence of 50 mM MgCl₂ (Control). Hybridization was also observed in the presence of MgCl₂ and spermine or spermidine. Furthermore, hybridization was also observed in the presence of spermine even when MgCl₂ was absent. The Conditions of FIG. 1 correspond to the ‘Millimolar concentration of cation in 2×MSS’ shown in Table 2. The second concentration in the labels of each column of FIG. 1 corresponds to the concentration of MgCl₂ shown in Table 2.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

What is claimed is:

1. A nucleic acid hybridization method, said method comprising:
obtaining a buffered solution comprising a double-stranded nucleic acid and a divalent cation;
converting said double-stranded nucleic acid to a single-stranded nucleic acid in said buffered solution, wherein said converting comprises digesting said double-stranded nucleic acid with a nuclease; and
hybridizing said single-stranded nucleic acid to a capture probe in said buffered solution.

2. The method of claim 1, wherein said double-stranded nucleic acid comprises DNA.

3. The method of claim 1, wherein said nuclease is selected from the group consisting of exonuclease III, T7 exonuclease and lambda exonuclease.

4. The method of claim 1, wherein said nuclease is lambda exonuclease.

5. The method of claim 1, wherein said buffer lacks a concentration of monovalent cations sufficient to substantially inhibit the activity of said nuclease.

6. The method of claim 1, wherein said buffer lacks monovalent cations.

7. The method of claim 1, wherein said buffer lacks a concentration of phosphate ions sufficient to substantially inhibit the activity of said nuclease.

8. The method of claim 1, wherein said buffer lacks phosphate ions.

9. The method of claim 1, wherein said divalent cation is present in said buffered solution at a concentration sufficient to permit hybridization of said single-stranded nucleic acid complementary to said capture probe.

10. The method of claim 1, wherein said divalent cation is selected from the group consisting of Mg²⁺, Mn²⁺, Zn²⁺ and combinations thereof.

11. The method of claim 1, wherein said buffered solution further comprises a polyamine.

12. The method of claim 1, wherein the pH of the buffered solution is at least 7.5.

13. The method of claim 12, wherein said buffered solution comprises Tris buffer.

14. The method of claim 1, wherein said capture probe is associated with a solid support.

15. The method of claim 14, wherein said solid support is a planar surface.

16. The method of claim 14, wherein said solid support is a microsphere.

17. The method of claim 16, wherein said microsphere is porous.

18. The method of claim 14, wherein said solid support is a fiber optic bundle.

19. The method of claim 1, wherein said capture probe is one of a plurality of capture probes.

20. The method of claim 19, wherein said plurality of capture probes is distributed on the surface of a substrate.

21. The method of claim 20, wherein said plurality of capture probes is orderly distributed.

22. The method of claim 20, wherein said plurality of capture probes is randomly distributed.

23. The method of claim 1 further comprising extending the 3′ end of said capture probe by providing a polymerase enzyme.

24. A method for detecting the presence of a nucleic acid complementary to a capture probe, said method comprising:
obtaining a buffered solution comprising a double-stranded nucleic acid and a divalent cation;
converting said double-stranded nucleic acid to a single-stranded nucleic acid in said buffered solution, wherein said converting comprises digesting said double-stranded nucleic acid with a nuclease;
providing said single-stranded nucleic acid to a capture probe in said buffered solution; and
determining whether said single-stranded nucleic acid hybridizes to said capture probe, wherein hybridization of said single-stranded nucleic acid to said capture probe indicates the presence of a nucleic acid complementary to said capture probe.

25. A hybridization composition comprising:
a solid support comprising a capture probe; and
a buffered solution in fluid communication with said capture probe, said buffered solution comprising a double-stranded nucleic acid, a divalent cation, and an nuclease for converting said double-stranded nucleic acid to a single-stranded nucleic acid.

26. A hybridization composition comprising:
a solid support comprising a capture probe; and
a buffered solution in fluid communication with said capture probe, said buffered solution comprising a nuclease, a single-stranded nucleic acid and a divalent cation present at a concentration sufficient to permit hybridization between the single-stranded nucleic acid and the capture probe provided that the single-stranded nucleic acid has sufficient complementarity to hybridize with the capture probe.
